(19) United States
(12) Patent Application Publication
(10) Pub. No.: US 2010/0160425 A1
(43) Pub. Date: Jun. 24, 2010

(54) ANTI-ALLERGIC AGENT AND BEVERAGE/FOOD, PREPARATION FOR EXTERNAL APPLICATION OR COSMETIC COMPRISING THE AGENT

(76) Inventors: Mari Yamamoto, Shizuoka (JP); Hiroshi Nagai, Ibaraki (JP)

Correspondence Address: KNOBBE MARTENS OLSON & BEAR LLP, 2040 MAIN STREET, FOURTEENTH FLOOR, IRVINE, CA, 92614, US

(21) Appl. No.: 12/160,554

(22) PCT Filed: Jan. 12, 2007
(86) PCT No.: PCT/JP2007/050322

§ 371 Date: Jul. 10, 2008

(30) Foreign Application Priority Data
Jan. 13, 2006 (JP) 2006-006521

(51) Int. Cl. A61K 31/352; C07D 311/04

(52) U.S. Cl. 514/456; 549/399

(57) ABSTRACT

The object is to isolate a substance having a potent anti-allergic effect inherent in tea leaves of an Assam hybrid line. Thus, disclosed is an anti-allergic agent comprising, as an active ingredient, a catechin yielded by the isolation, i.e. epicatechin-3-O-(3-O-methyl)gallate, epicatechin-3-O-(4-O-methyl)gallate, epicatechin-3-O-(3,4-O-dimethyl)gallate, epicatechin-3-O-(3,5-O-dimethyl)gallate, epicatechin-3-O-(3,4,5-O-tromethyl)gallate, an epimer thereof or the like. Also disclosed is a food/beverage, a preparation for external application, and a cosmetic including the anti-allergic agent. An anti-allergic composition can be prepared using a compound represented by the general formula (1):

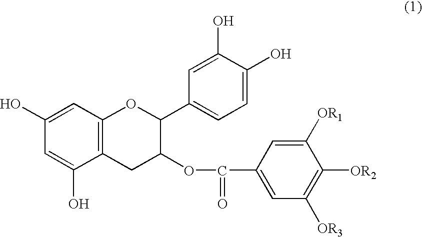

in which R₁, R₂ and R₃ independently represent a hydrogen atom or a methyl group.

### TECHNICAL FIELD

The present invention relates to an anti-allergic agent containing a novel catechin as an active ingredient and a food/beverage, a preparation for external application, and a cosmetic containing the anti-allergic agent.

### BACKGROUND ART

In modern societies, the diversification of eating habits and life styles has progressed, while allergic symptoms have also diversified and increased yearly. Nowadays, the number of patients with allergies, including potential patients, is said to be 30 million. Furthermore, the side effects of medical agents, such as steroids, are of concern. Even while being aware of their allergic symptoms, some people fear the side effects of medical agents and cannot voluntarily receive medical treatment, which causes them to be afflicted with symptoms of allergies. Therefore, consumers have had high interest and expectations in the development of food/beverages having a component with an anti-allergic action that can be easily ingested without anxiety.

It has been reported that catechins, saponin, flavonoid and caffeine contained in green tea have an anti-allergic effect (see Patent Document 1, Non-patent Documents 1 and 2), methylated catechin containing green teas such as Benifuuki, Benifuji, and Benihomare have I-type allergy-inhibiting effect and anti-inflammatory effect (see Patent Document 2).

Among them, green teas or pouchong teas made from Assam hybrid, such as Benifuuki, Benifuji, and Benihomare, have an anti-allergic effect and an anti-inflammatory effect, and the utilization of the teas is being planned. However, the leaves of these teas are currently manufactured in small qualities, and compared to their high price, their effectiveness varies between individuals. Therefore, a food/beverage which can be expected to have an effect for everyone has been sought.

Patent Document 1: Japanese Unexamined Patent Application, First Publication No. 2001-253879

Patent Document 2: Japanese Unexamined Patent Application, First Publication No. 2000-159670

Non-patent Document 1: Allergy, 52,58 (1997), Fragrance J., 11, 50 (1990)

Non-patent Document 2: Biol. Pharm. Bull., 20, 565 (1997)

### DISCLOSURE OF THE INVENTION

### Problems to be Solved by the Invention

As catechins having anti-allergic activity, Epigallocatechin-3-O-(3-O-methyl)gallate (hereinafter, referred to as EGCG3″Me) has been known. However, although this EGCG3″Me is also contained in other leaves of green tea, the strong anti-allergic activity of extracts from the tea leaves of the above-described Assam hybrid has so far not been explained only by the anti-allergic activity of EGCG3″Me.

If a substance having high anti-allergic activity inherent to tea leaves of Assam hybrid could be isolated and used as an anti-allergic agent, a food/beverage which could be expected to have an effect for everyone could be easily manufactured. However, thus far, it has not been understood what substance has the high anti-allergic activity inherent to tea leaves of Assam hybrid.

In consideration of the above-mentioned problem, the present invention aims at the isolation of a substance having a high anti-allergic activity inherent to tea leaves of Assam hybrid, and provides an anti-allergic agent containing, as an active ingredient, obtained catechins, i.e. epictechin-3-O-(3-O-methyl)gallate, epictechin-3-O-(4-O-methyl)gallate, epictechin-3-O-(3,4-O-dimethyl)gallate, epictechin-3-O-(3,5-O-dimethyl)gallate, epictechin-3-O-(3,4,5-O-trimethyl)gallate and epimer thereof, and a food/beverage, a preparation for external application or a cosmetic containing the substance.

### Means for Solving the Problems

More specifically, the present invention provides the following.

In a first aspect, a composition for producing an anti-allergic agent is represented by following general formula (1):

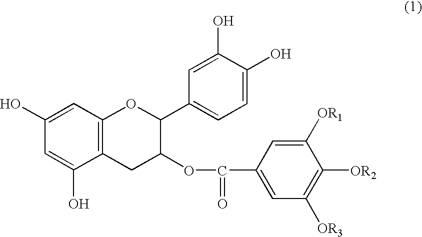

in which R₁, R₂ and R₃ independently represent one of a hydrogen atom and a methyl group.

As described above, catechins have various effects, such as an antioxidant action, an inhibitory effect on arteriosclerosis, an inhibitory effect on high blood pressure, an inhibitory effect on elevated blood glucose, an antiseptic action, an antibacterial action, and an odor eliminating action. The present inventors have found that the compound represented by the general formula (1), i.e. epictechin-3-O-(3-O-methyl)gallate (hereinafter, referred to as ECG3″Me), epictechin-3-O-(4-O-methyl)gallate (hereinafter, referred to as ECG4″Me), epictechin-3-O-(3,4-O-dimethyl)gallate (hereinafter, referred to as ECG3″4″diMe), epictechin-3-O-(3,5-O-dimethyl)gallate (hereinafter, referred to as ECG3″5″diMe), epictechin-3-O-(3,4,5-O-trimethyl)gallate (hereinafter, referred to as ECG3″4″5″triMe) and epimer thereof (hereinafter, referred to as CG3″Me, CG4″Me, CG3″4″diMe, CG3″5″diMe, CG3″4″5″triMe), have the high anti-allergic activity inherent to tea leaves of Assam hybrid. Among them, the ECG3″Me was found to have an especially strong anti-allergic activity. These substances are easily epimerized when extracting the tea leaves, and thereby, for example, when an isolating means such as chromatography is used, these substances are isolated with other methylated catechins. Meanwhile, the relationship between R₁, R₂ and R₃ is as follows.

TABLE 1

| | R₁ | R₂ | R₃ |
|---|---|---|---|
| ECG3″Me (CG3″Me) | Methyl | Hydrogen | Hydrogen |
| | Group | Atom | Atom |
| ECG4″Me (CG4″Me) | Hydrogen | Methyl | Hydrogen |
| | Atom | Group | Atom |
| ECG3″4″diMe (CG3″4″diMe) | Methyl | Methyl | Hydrogen |
| | Group | Group | Atom |
| ECG3″5″diMe (CG3″5″diMe) | Methyl | Hydrogen | Methyl |
| | Group | Atom | Group |
| ECG3″4″5″triMe | Methyl | Methyl | Methyl |
| (CG3″4″5″triMe) | Group | Group | Group |

Therefore, this compound is used as a compound for producing the anti-allergic agent to provide an anti-allergic agent having a more rapid effect. In addition, because this compound is one kind of catechin extracted from tea leaves, there is little concern about such side effects as seen in pharmaceutical agents.

An “anti-allergic agent” referred to herein indicates an agent having ECG3″Me and the like as an active ingredient, and achieving an effect of inhibiting allergic symptoms. It is sufficient that the ECG3″Me and the like in the anti-allergic agent is included to the extent that the agent is judged to achieve a sufficient effect, that is, an amount as an active ingredient. Thus, in the present invention, an anti-allergic agent includes both an ECG3″Me simple substance, and mixture further containing other catechins, preservatives, antiseptic agents and the like.

In a second aspect, a composition for producing the anti-allergic agent described in the first aspect is an ingredient derived from tea leaves of one of green tea and pouchong tea, raw materials of which are leaves of Assam hybrid.

The ECG3″Me and the like represented by the general formula (1) are compositions derived from leaves of Assam hybrid. Therefore, according to the second aspect of the present invention, leaves of Assam hybrid are made to be raw materials of green tea or pouchong tea to increase the amount of the ECG3″Me and the like contained in the tea leaves, whereby the composition for producing the anti-allergic agent can be efficiently produced.

In a third aspect of the composition for producing the anti-allergic agent described in the second aspect, the leaves of the Assam hybrid are Benifuuki.

Benifuuki has an especially strong anti-allergic activity among tea leaves of the Assam hybrid line. Thus, according to the third aspect of the present invention, leaves of Benifuuki are made to be raw materials of green tea or pouchong tea to increase the amount of ECG3″Me and the like contained in tea leaves, and therefore the ECG3″Me and the like can be more efficiently isolated. In addition, a phosphating solution, acetic acid solution, citric acid solution or ascorbic acid solution can be added upon isolating. The decomposition of hydrolyzable tannin, such as strictinin, can be prevented by adding an acid solution such as a phosphating solution. Furthermore, the addition of such a solution can improve the extraction efficiency of catechins.

According to a fourth aspect, in a composition for producing the anti-allergic agent according to any one of the first to third aspects, under a condition in which a measurement is carried out by high-performance liquid chromatography using an octadecyl silica column having a particle size of 5 μm and a column length of 150 mm, and the eluent is a mixture of water, acetonitrile, phosphoric acid, and methanol, the composition for producing the anti-allergic agent is a component which moves at an elution time between 37 minutes to 50 minutes of a polyphenolic fraction based on a flow rate of the mobile phase of 1 ml/min.

According to the fourth aspect of the present invention, a composition isolated by an eluent being a mixture of water, acetonitrile, phosphoric acid, and methanol using an octadecyl silica column having a particle size of 5 μm and a column length of 150 mm (ODS-C18 column), based on a flow rate of the mobile phase of 1 ml/min, is used as a composition for producing the anti-allergic agent, and therefore the composition for producing the anti-allergic agent can be produced more efficiently.

An eluent, in which each of the water, acetonitrile, phosphoric acid, and methanol are mixed together at a predetermined ratio, is used. For example, it is preferable that a water, acetonitrile, and phosphoric acid mixture in a 400:10:1 volume ratio, respectively, is designated as eluent A, and the eluent A and a methanol mixture in a 2:1 volume ratio is used as eluent B.

In a fifth aspect, a beverage contains the composition for producing the anti-allergic agent according to any one of the first to fourth aspects in an amount of 0.08 mg to 80 mg per 100 ml of the beverage.

According to the fifth aspect of the present invention, the content of the composition for producing the anti-allergic agent in the beverages is the above-described amount, so that the catechins can provide an anti-allergic action in the beverages. If the content is less than 0.08 mg, sufficient anti-allergic action cannot be provided. If the content is more than 80 mg, flavor is lost.

In addition, “beverage” used in the present invention indicates a wide variety of beverages, e.g., green tea, black tea, tea drinks such as Chinese tea, soft drinks such as fruit juice drinks and a sports drinks, coffee, cocoa, juices, milk drinks, carbonated beverages, alcoholic beverages, and other various beverages listed in the Japan standard commodity classification (Management and Coordination Agency).

According to a sixth aspect, a beverage as described in the fifth aspect is a beverage contained in an airtight container.

According to the sixth aspect of the present invention, the beverage is contained in an airtight container so that it can be readily ingested. As a result, everyday behavior, such as drinking tea, can prevent and decrease allergic symptoms.

In addition, “beverage contained in an airtight container” in the present invention indicates beverages filled into a molded container, a main component of which is polyethylene terephthalate (so called PET bottle), a metal can, a paper container in which metal foil and plastic film are combined, a jar, and the like.

In a seventh aspect, a food product contains the composition for producing the anti-allergic agent according to any one of the first to fourth aspects in an amount of 0.08 mg to 80 mg per 100 ml of the food product.

According to the seventh aspect of the present invention, the content of the composition for producing the anti-allergic agent in the food products is the above-described amount, and thereby it may be possible for catechins to provide an anti-allergic action in the food products. If the content is less than 0.08 mg, sufficient anti-allergic action cannot be provided. If the content is more than 80 mg, flavor is lost.

In addition, the “food products” in the present invention are not especially limited as long as their configuration can include the composition for producing the anti-allergic agent according to the present invention. For example, the food products include solid foods such as breads, cookies, chocolates, chewing gums, cereals, and sweets, and gel food products or semi-solid food products such as jams, yogurts, and jellies.

In an eighth aspect, a method is provided which uses the composition for producing the anti-allergic agent according to any one of the first to fourth aspects as an anti-allergic enhancer.

According to the eighth aspect of the present invention, the composition for producing the anti-allergic agent is added to, as an anti-allergic enhancer, tea drinks having a low content of the ECG3″Me and the like, such as Yabukita, and to food products developed using an anti-allergic action of cereal tea and mix tea, so that the anti-allergic activity can be enhanced without impairing the functions which these tea drinks inherently possess.

In addition, “anti-allergic enhancer” used in the invention includes, besides ones obtained by purifying the composition represented by the general formula (1), for example, a mixture in which gallocatechin gallate, catechin, and the like are adequately mixed as necessary. Forms such as liquid, powder, and particles pose no problem.

In a ninth aspect, a method for producing a composition for producing an anti-allergic agent is provided which includes the steps of: adding a solvent to a tea leaf powder of green teas or pouchong teas made from Assam hybrid leaves to obtain a mixture; and extracting a composition for producing an anti-allergic agent represented by the following general formula (1) from the mixture to purify the mixture by high-performance liquid chromatography:

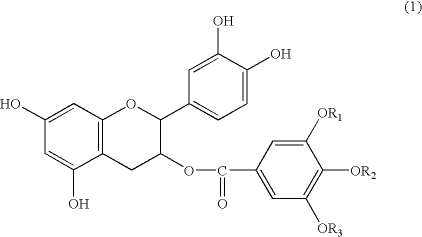

in which R₁, R₂ and R₃ independently represent a hydrogen atom or a methyl group.

In a tenth aspect, a method is provided using an octadecyl silica column having an inner diameter of 4.6 mm, a length of 150 mm, and particle size of 5 μm to produce a composition for producing an anti-allergic agent represented by the following general formula (1):

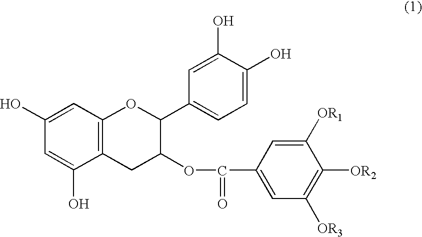

in which R₁, R₂ and R₃ independently represent a hydrogen atom or a methyl group.

### EFFECTS OF THE INVENTION

According to the present invention, the ECG3″Me and the like isolated from leaves of Assam hybrid are used as a composition for producing the anti-allergic agent, so that an anti-allergic agent having an improved fast-acting property can be provided. Furthermore, the composition for producing the anti-allergic agent is derived from tea leaves, and therefore concerns about inducing side effects such as drowsiness are low. Therefore, anybody can ingest the composition with confidence.

Foods/beverages having a flavor suitable to everyone can be provided by adding the composition to the foods/beverages. This can allow the composition to be taken by an ordinary behavior such as drinking beverages and eating food products, resulting in the prevention of allergic symptoms.

Moreover, the composition is used as an anti-allergic enhancer, so that an anti-allergic activity can be enhanced without impairing the inherent function of tea drinks and the like having a low anti-allergic effect.

### BRIEF DESCRIPTION OF THE DRAWINGS

FIG. 1 is a drawing showing a result of an isolation of an anti-allergic agent according to the present invention using chromatography;

FIG. 2 is a drawing showing a result in which the inhibitive effect on histamine release is investigated by varying the amount of addition of catechins;

FIG. 3 is a drawing showing the relationship between the mixture ratio of EGCG3″Me and ECG3″Me and the inhibitive effect on histamine release; and

FIG. 4 is a drawing showing the relationship between additive amount of EGCG3″Me and ECG3″Me and the inhibitive effect on histamine release.

### PREFERRED MODE FOR CARRYING OUT THE INVENTION

Hereinafter, the present invention is described in detail; however, the present invention is not limited thereto.

### (Production of Tea Leaf Extract)

A method for producing a composition for producing an anti-allergic agent according to the present invention includes the steps of: adding a solvent to tea leaf powders of green teas or pouchong teas made from Assam hybrid leaves, especially, Benifuuki green tea or Benifuuki pouchong tea to obtain a mixture; and extracting a composition for producing an anti-allergic agent represented by the following general formula (1) from the mixture to purify the mixture by high-performance liquid chromatography. Hereinafter, each step where Benifuuki leaf is used will be described below sequentially:

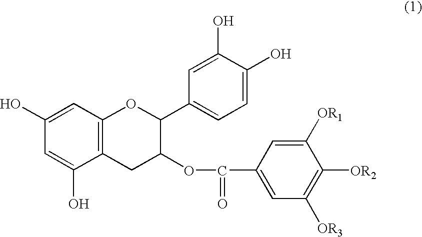

in which R₁, R₂ and R₃ independently represent a hydrogen atom or a methyl group.

First, in the step of obtaining the mixture, Benifuuki leaves may be in leaf form, but are preferably in powder form. Furthermore, the size of the crushed material is preferably uniform, and the crushed material may be used after sieving.

In addition, an extraction solvent may be one which has no toxicity, and the solvent may be water and low alcohols, for example, methanol, ethanol, propanol, isopropanol, butanol, ethers such as isobutanol, for example, ethyl ether, dioxane, acetone and the like. Using a solvent containing ethanol is preferable when considering the collection rate of catechins in the extract. The temperature of the extraction solvent is not especially limited as long as it is higher than the melting point, and lower than the boiling point. It is desirably 10° C. to 100° C. for water, 10° C. to 40° C. for ethanol and methanol. The extracting time is preferably 10 seconds to 24 hours.

For extraction, at first, the solvent is added to 250 mg of tea leaves or tea leaf powders to obtain a mixture. At this time, a phosphating solution can be added to increase the extraction efficiency. The concentration of the phosphating solution is preferably 0.1% to 5%, and more preferably 0.8%. Next, an extraction solvent such as ethanol and water is added to the mixture, and incubation is carried out at 20° C. to 40° C. for 15 minutes to 120 minutes.

### (Isolation of ECG3″Me and the Like)

The step of purification (isolation) by high-performance liquid chromatography is a step in which the isolation is carried out for the mixture obtained by the above method, using a method generally used as a chemical separation purification method. As a chemical separation purification method, for example, liquid-liquid separation, thin layer chromatography, adsorption column chromatography, partition column chromatography, gel filtration chromatography, ion exchange chromatography, cataphoresis, high performance liquid chromatography and the like can be used. These separation and purification means can be combined, if necessary. The ECG3″Me isolated by the aforementioned method and its isomerized form and additives usually added to an anti-allergic agent, such as preservatives and antiseptic agent, are collectively included in the anti-allergic agent according to the present invention.

Isolation is preferably carried out using high-performance liquid chromatography (HPLC) in light of ease of operation, good separation capacity, and the like. In this case, isolation is carried out according to the following sequence.

First, the above-described mixture is diluted with distilled water followed by being stirred, then left at rest for a few minutes, and filtrated. It is preferable that the filtrate is collected in a falcon tube. This filtrate is filtrated by a hydrophilic filter and collected in an eppen tube. Furthermore, the supernatant is diluted ten times with the distilled water. Then, the resultant is isolated under the following condition.

It is preferable that an eluent A (mobile phase A) is adjusted with water, acetonitrile, and phosphoric acid (H₃PO₄) in an 800:10:1 to 400:40:1 volume ratio, and more preferably 400:10:1. On the other hand, it is preferable that an eluent B (mobile phase B) is adjusted with methanol and the eluent A mixture in a 1:1 to 1:4 volume ratio, more preferably 1:2.

Isolation is preferably performed under the following conditions. Firstly, the flow rate of the mobile phase is set to 1 ml/min, and a linear gradient is run to 20% by mass of the eluent B until 3 minutes after the start of the separation (the start of the measurement), to 75% by mass of the eluent B by 30 minutes after the start of separation. Then, this condition is kept until 45 minutes after the start of separation, and the eluent B is lowered at once to 20% by mass.

A commercially-available column is usually used; however, a new column is preferable, when possible. As shown in Table 2, this is because, along with deterioration of the column, the ECG3″Me and its isomerized form are likely to be isolated together with GCG3″Me.

TABLE 2

| | | Column After One |
|---|---|---|
| | New Column | Month of Usage |
| EGCG3″Me Area | 245973 | 252558 |
| GCG3″Me Area | 283716 | 378950 |
| ECG3″Me Area | 88023 | 0 |
| GCG3″Me/EGCG3″Me | 1.153 | 1.500 |
| (GCG3″Me + ECG3″Me)/ | 1.511 | 1.500 |
| EGCG3″Me | | |

In addition, in the present invention, a concentrate of the ECG3″Me and the like isolated by the above described method may be used as an anti-allergic agent. The concentration is carried out by an ordinarily-performed method, for example, by a method using a rotary evaporator under reduced pressure. The temperature at this time is preferably 20° C. to 80° C., and more preferably no greater than 60° C.

### (Manufacture of Food/Beverage)

The composition for producing the anti-allergic agent according to the present invention may be used as an anti-allergic agent in which the composition is used alone or together with other catechins for various applications such as a beverage, a medical drug, and food product. As a food product, the composition can be mixed in a food for specified health use, a special nutritious food, a dietary supplement, a health food and the like as a food additive. Various kinds of food products can be target food products to have the composition added. As a beverage, the composition can be mixed in beverages designated as foods for specified health uses, special nutritious foods, dietary supplements, and other nutritional beverages, health beverages, various kinds of health teas, other beverages and the like. Other food products include sweets, breads, noodles, soybean processed foods, dairy products, egg processed foods, paste products, oils and fats, seasonings, and the like.

Known production methods can be used as a concrete production method. In addition, crushed objects in which tea leaves themselves are crushed may be further added during a production process. Other methylated catechins synthesized biochemically may also be mixed.

“Methylated catechins” used herein indicate methylated catechins and inevitable components produced during purification. Methylated catechins according to the present invention correspond mainly to epigallocatechin-3-O-(3-O-methyl)gallate (hereinafter, referred to as EGCG3″Me), epigallocatechin-3-O-(4-O-methyl)gallate (hereinafter, referred to as EGCG4″Me), gallocatechin-3-O-(3-O-methyl)gallate (hereinafter, referred to as GCG3″Me), or gallocatechin-3-O-(4-O-methyl)gallate (hereinafter, referred to as GCG4″Me), in addition to epictechin-3-O-(3-O-methyl)gallate (hereinafter, referred to as ECG3″Me), epictechin-3-O-(4-O-methyl)gallate (hereinafter, referred to as ECG4″Me), epictechin-3-O-(3,4-O-dimethyl)gallate (hereinafter, referred to as ECG3″4″diMe), epictechin-3-O-(3,5-O-dimethyl)gallate (hereinafter, referred to as ECG3″5″diMe), epictechin-3-O-(3,4,5-O-trimethyl)gallate (hereinafter, referred to as ECG3″4″5″triMe), catechin-3-O-(3-O-methyl)gallate (hereinafter, referred to as CG3″Me), catechin-3-O-(4-O-methyl)gallate (hereinafter, referred to as CG4″Me), catechin-3-O-(3,4-O-dimethyl)gallate (hereinafter, referred to as CG3″4″diMe), catechin-3-O-(3,5-O-dimethyl)gallate (hereinafter, referred to as CG3″5″diMe), and catechin-3-O-(3,4,5-O-teimethyl)gallate (hereinafter, referred to as CG3″4″5″triMe).

Moreover, when the composition for producing the anti-allergic agent according to the present invention is used for a food/beverage and preparation for external application by mixing it with other catechins, it is preferable that they are mixed so that the value of EGCG3″Me+GCG3″Me)/(ECG3″Me+CG3″Me) is 0.5 to 6. When the numeric value is less than 0.5, sufficient anti-allergic effect cannot be obtained. When the numeric value is more than 6, flavor may be lost.

In addition, in order for the above-described composition for producing the anti-allergic agent to give sufficient anti-allergic effect in beverages and food products, antioxidants, fragrances, various kinds of esters, organic acids, organic acid salts, inorganic acids, inorganic acid salts, inorganic salts, pigments, emulsions, preservatives, seasonings, sweeteners, acidulants, fruit juice extracts, vegetable extracts, floral nectar extracts, pH adjusters, quality stabilizers and the like may be mixed alone or in combinations thereof.

For example, the sweetener includes sugar, glucose, fruit sugar, isomerized syrup, glycyrrhizine, stevia, aspartame, fructooligosaccharide, galacto-oligosaccharide, and the like. The acidulant includes a citric acid, a tartaric acid, a malic acid, an lactic acid, a fumaric acid, and a phosphoric acid, in addition to fruit juices extracted from natural ingredients. A citric acid or a malic acid is contained in a beverage in an amount of 0.1 g/L to 5 g/L, or preferably 0.5 g/L to 2 g/L. An antioxidant includes L-ascorbic acid, L-sodium ascorbate, erythorbic acid, and sodium erythorbate. An antioxidant is 0.005% to 0.5% by mass, or more preferably 0.01% by mass in a beverage.

A container used for a beverage can be provided in the form of, similar to a general beverage, molded containers, a main component of which is polyethylene terephthalate (so called PET bottle), metal cans, paper containers in which metal foil and plastic film are combined, and jars.

Furthermore, among the aforementioned containers, for example, metal cans, which can be sterilized by heat after a beverage is filled, are manufactured under predetermined sterilization conditions defined under food hygiene laws. As for PET bottles and paper containers, which cannot be sterilized, a method, in which a container is sterilized under similar conditions as described above, such as sterilization by a plate type heat exchanger under high temperature for a short time, followed by cooling the container to a certain temperature and filling it with a beverage, is employed. Alternatively, a filled container may be filled with other ingredients under antiseptic conditions. Moreover, an operation in which thermal sterilization is carried out under acidic conditions, and then the pH is returned to neutral under an aseptic condition, or thermal sterilization is carried out under neutral conditions, and then the pH is returned to acidic conditions under aseptic conditions, can be carried out.

When filling transparent containers such as PET bottles, it is preferable that an antioxidant, such as L-ascorbic acid and L-sodium ascorbate, is added to prevent browning.

Medical drugs having the composition for producing the anti-allergic agent according to the present invention include medical drugs used to treat allergic coryza, atopic dermatitis, asthma, hives and hyperlipidemia, adipositas, hepatic disease, and hyperpiesia.

### (Use as Anti-Allergic Enhancer)

The composition for producing the anti-allergic agent according to the present invention can be used for a tea drink having a low ECG3″Me content, such as Yabukita tea, cereal tea, mix tea and the like as an anti-allergic enhancer. Thus, it may be possible to enhance the anti-allergic effect of catechins contained in the tea drinks.

In a concrete method for the case of adding to Yabukita tea, a tea leaf extract is produced using the aforementioned method, and ECG3″Me and the like are isolated therefrom. Then, the isolated composition for producing the anti-allergic agent is added to Yabukita tea. A preferable additive amount is 0.08 mg to 80 mg per 100 ml of beverage, and more preferably 0.4 mg to 40 mg per 100 ml of beverage.

### (Manufacture of Pharmaceutical Agent)

Concerning a pharmaceutical agent, the composition for producing the anti-allergic agent can be administered as it is, or by dilution with water and the like. Alternatively, a pharmaceutical agent is prepared by formulating with known pharmaceutical carriers. For example, the composition can be administered as oral liquid formulations such as syrup, or oral solid formulation in the form of tablets, capsules, granules, powdered medicine and the like by processing into an extract, powder, and the like, and blending with pharmaceutically acceptable carriers. As a pharmaceutically acceptable carrier, various organic or inorganic carrier substances generally used as pharmaceutical formulations are used, and the carriers are blended as diluents, lubricants, binders and disintegrants for solid formulations, and solvents, diluents, suspending agents, binders and the like for liquid formulations. Formula additives such as antiseptic agents, antioxidants, coloring agents, sweetening agents and the like can be used as necessary.

In addition to the above internal medicines, the composition can be used as a preparation for external application by adding to semi-solid materials such as ointments, gel agents, gel agents, creams, pack agents, and emulsions, liquids such as lotion agents, toners, and solid materials such as powders. These preparations for external application can be manufactured using conventionally known methods, and it is preferable that the temperature upon mixing a base and the composition for producing the anti-allergic agent according to the present invention is 20° C. to 80° C., and more preferably no greater than 50° C. Furthermore, the additive amount of the composition for producing the anti-allergic agent is preferably 0.1% to 3% by mass, and more preferably 0.2% to 2% by mass.

### Examples

### Production of Anti-Allergic Agent

10 ml of a 2% phosphating solution was added to 250 mg of Benifuuki leaf powder, the water content of which was measured in advance (water content 3%), to obtain a tea leaf dispersion liquid. Then, 10 ml of ethanol was added to this tea leaf dispersion liquid, stirred, and further incubated at 30° C. for 60 minutes. Next, the resultant was diluted with distilled water followed by stirring, left at rest for approximately 5 minutes, and then filtrated. 10 ml of filtrate was collected in a 15 ml falcon tube. This filtrate was filtrated by a 0.45 μm hydrophilic filter (manufactured by ADVANTEC, INC.: DISMIC-13HP, PTFE non-sterile), and collected in a 1.5 ml eppen tube. The supernatant was further diluted to ten times with distilled water (900 μl of distilled water and 100 μl of supernatant in a 1.5 ml eppen tube). Next, approximately 100 μl of the resultant was then collected in an auto-injector tube, ECG3″Me was isolated under the following conditions, and the anti-allergic agent of the present invention was obtained.

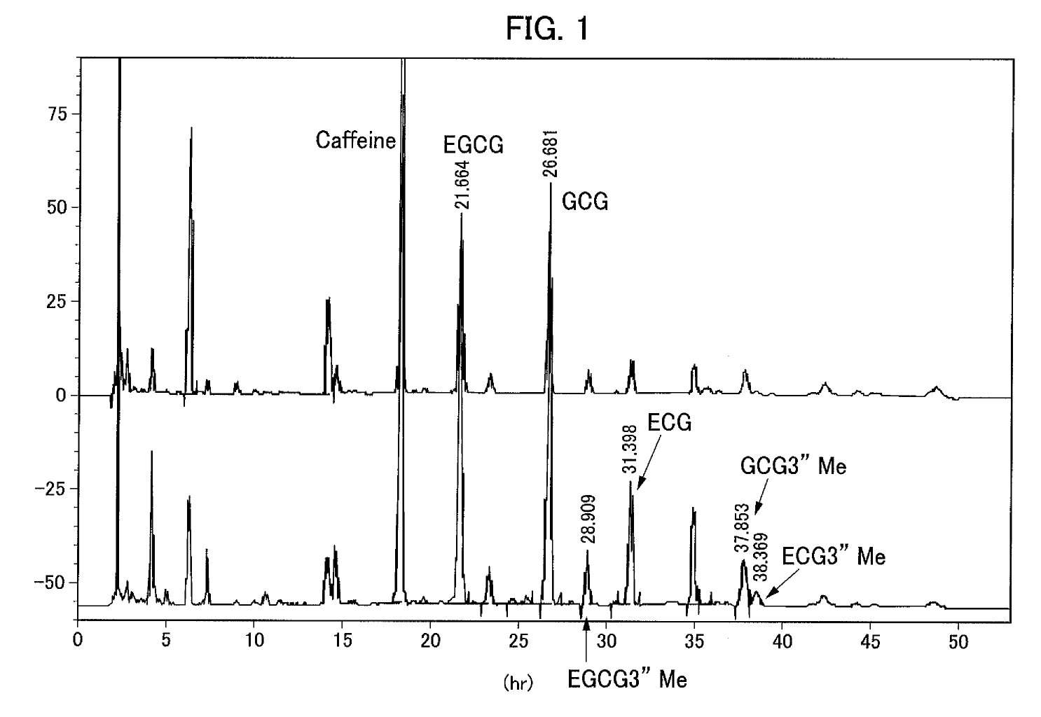

A chromatography chart upon isolation of ECG3″Me is shown in the lower part of FIG. 1. In FIG. 1, the chart in the upper part is a chart of a tea leaf dispersion liquid, in which the content of ECG3″Me in the tea leaf is low. This showed that ECG3″Me and the like was isolated.

Mobile phase A: water, acetonitrile, and phosphoric acid mixture (volume ratio 400:10:1)

Mobile phase B: mixture of methanol and mobile phase A (volume ratio 1:2)

Column: Wako Pure Chemical (Wakopak Navi C18-5 (4.6×150) 5 μm)

Guard Column: Wako Pure Chemical (Wakopak Navi C18-5 (4.6×10) 5 μm)

Auto Sampler: 4° C.

Column temperature: 40° C., Injection rate: 20 μl, Flow rate: 1 ml/min

Detection wavelength: 272 nm (detected by UV-VIS or PDA detector)

### (Investigation on Histamine Release Inhibiting Effect of ECG3″Me)

The histamine release inhibiting effect of the anti-allergic agent obtained by the abovementioned method was investigated. In addition, as a comparison sample, isolations of other catechins, epigallocatechin-3-O-gallate (hereinafter, referred to as EGCG), and epigallocatechin-3-O-(3-O-methyl)gallate (hereinafter, related to as EGCG3″Me) were used.

In order to determine the anti-allergic activity (type I allergy), inhibitory activity on the release of histamine from mouse mast cells was used as an indicator. The histamine release inhibitory effect was investigated by using mouse bone marrow-derived cultured mast cells (BMMC). The cells were cultured in a RPMI1640 medium supplemented with 10% heat inactivated FBS (fetal bovine serum), 3 ng/ml of interleukin-3 (IL-3), 5 mM sodium glutamate, and 50 μM 2-mercaptoethanol.

The cells (1×10⁷ cells/ml) were sensitized anti-DNP mouse IgE antibody overnight, then, on the following day, suspended in a Tyrode solution and incubated with the test samples for 10 minutes, degranulation was induced by adding DNP-HSA (antigen) (20 minutes), and the amount of histamine in the supernatant fluid was determined using liquid chromatography.

An LC-10A (manufactured by Shimadzu Corporation) was used as the measuring instrument, and asahipak-Shodex ODP 50-4E was used as the column. Isocratic analysis was conducted under the following conditions: flow rate of 0.5 ml/min; injection amount of 20 μl; temperature of the column oven of 37° C.; detector RF (e.g., 330 nm, em. 430 nm). The anti-allergic activity was determined to be high when the relative value to distilled water, which was a control, was lower.

The results are shown in Table 3. These results show that the composition for producing the anti-allergic agent according to the present invention has a stronger histamine release inhibiting effect compared to other catechins.

TABLE 3

| | Ratio of Histamine Release |
|---|---|
| Sample | Inhibiting Effect % |
| EGCG 10 μg/ml | 7.63 |
| EGCG3″Me 10 μg/ml | 29.6 |
| EGG 3″Me 10 μg/ml | 46.79 |

### (Investigation of Effect of Additive Amount of ECG3″Me on Histamine Release Inhibiting Effect)

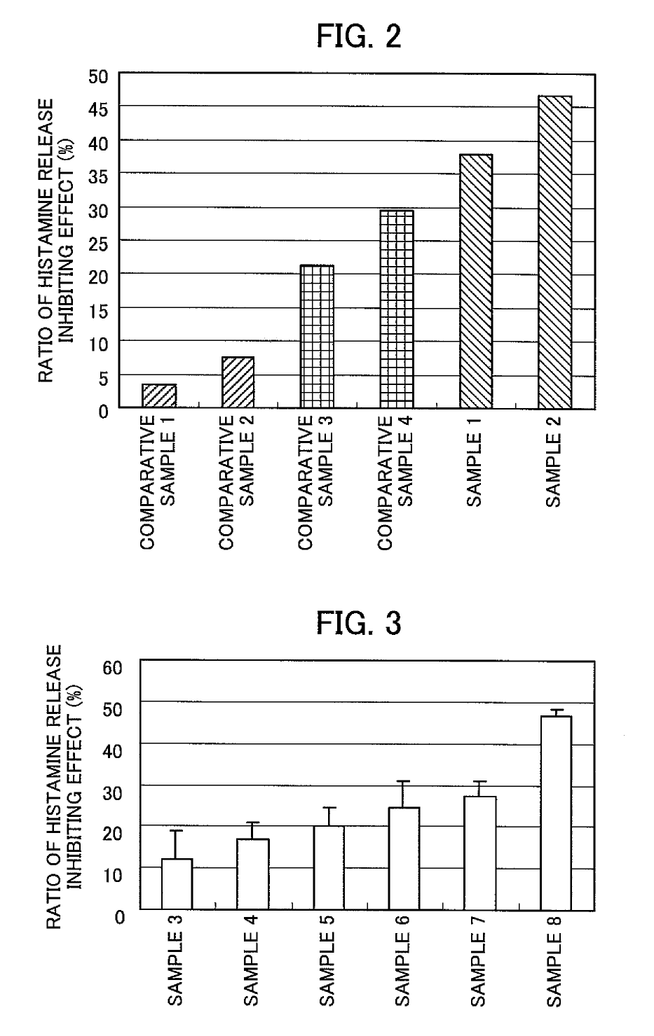

Next, the histamine release inhibiting effect was investigated by varying the additive amount of the above catechins. Beverages containing ECG3″Me in the amount of 2 μg/ml and 10 μg/ml were designated as Samples 1 and 2, respectively. In addition, beverages containing EGCG in the amount of 2 μg/ml and 10 μg/ml were designated as Comparative Samples 1 and 2, respectively. Beverages containing EGCG3″Me in the amount of 2 μg/ml and 10 μg/ml were designated as Comparative Samples 3 and 4, respectively. The histamine release inhibiting effects of these samples are shown in FIG. 2. These results show that the composition for producing the anti-allergic agent according to the present invention has a histamine release inhibiting effect of approximately 1.5 times that of EGCG3″Me.

### (Investigation on Relationship Between Mixture Ratio of EGCG3″Me and ECG3″Me, and Histamine Release Inhibiting Effect)

Next, the relationship between the mixture ratio of EGCG3″Me and ECG3″Me, and the histamine release inhibiting effect was investigated. Furthermore, the anti-allergic activity of the extract extracted from Benifuuki by hot water was also investigated. The catechin content in the samples was adjusted to be the amount as shown in the following Table 4, and the histamine release inhibiting effect was investigated. The results are shown in FIG. 3. For the mixture of EGCG3″Me and ECG3″Me, the strength of the anti-allergic activity was estimated to be from 1.5 to 2.5. In the investigation, Benifuuki green tea hot-water extract is shown to have stronger activity than the individual substances.

TABLE 4

| | |
|---|---|
| Sample 3 | EGCG3″Me: 50 μg + ECG3″Me: 0 μg |
| Sample 4 | EGCG3″Me: 37.5 μg + ECG3″Me: 12.5 μg |
| Sample 5 | EGCG3″Me: 25 μg + ECG3″Me: 25 μg |
| Sample 6 | EGCG3″Me: 12.5 μg + ECG3″Me: 37.5 μg |
| Sample 7 | EGCG3″Me: 0 μg + ECG3″Me: 50 μg |
| Sample 8 | Benifuuki Hot Water Extract: 50 μg/ml |

### (Investigation on Relationship of Additive Amount of EGCG3″Me and ECG3″Me, and Histamine Release Inhibiting Effect)

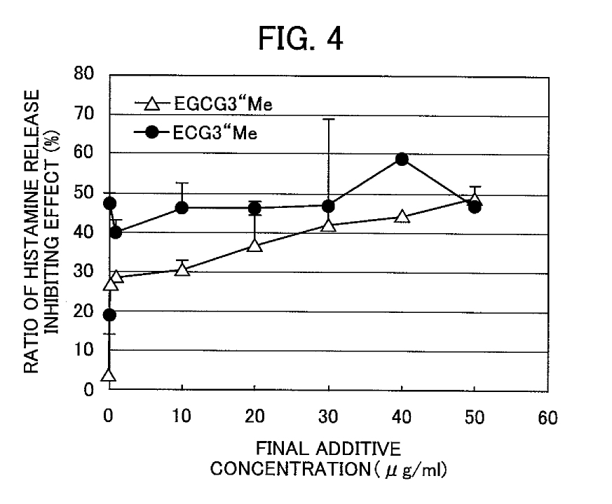

The histamine release inhibiting effect of respective cases where EGCG3″Me or ECG3″Me was added to Yabukita hot-water extract having low anti-allergic activity, to have a final additive concentration of 0.1 μg/ml, 1μ μg/ml, 10 μg/ml, 20 μg/ml, 30 μg/ml, 40 μg/ml, 50 μg/ml, respectively, was investigated, and the results are shown in FIG. 4. This shows that ECG3″Me can increase the anti-allergic activity more than EGCG3″Me. As described above, it is shown that a ECG3″Me derivative extracted from Assam hybrid can provide an anti-allergic activity by being added to tea leaves having less anti-allergic activity.

1. A composition for producing an anti-allergic agent represented by the following general formula (1): 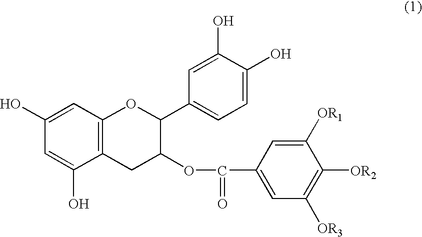
wherein R₁, R₂ and R₃ independently represent one of a hydrogen atom and a methyl group.

2. The composition for producing the anti-allergic agent according to claim 1, wherein the composition is an ingredient derived from leaves of one of green tea and pouchong tea, a raw material of which is leaves of Assam hybrid.

3. The composition for producing the anti-allergic agent according to claim 2, wherein the leaves of Assam hybrid are Benifuuki.

4. The composition for producing the anti-allergic agent according to any one of claims 1 to 3, wherein, under conditions in which a measurement is carried out by high-performance liquid chromatography using an octadecyl silica column having a particle size of 5 μm and a column length of 150 mm, the composition is such that an eluent is a mixture of water, acetonitrile, phosphoric acid, and methanol, and a component moves at an elution time in the range of 37 minutes to 50 minutes of a polyphenolic fraction under the condition that a flow rate of a mobile phase is 1 ml/min.

5. A beverage comprising the composition for producing the anti-allergic agent according to claim 1, in an amount in a range of 0.08 mg to 80 mg per 100 ml of the beverage.

6. The beverage according to claim 5, wherein the beverage is contained in an airtight container.

7. A food product comprising the composition for producing the anti-allergic agent according to claim 1 in an amount of 0.08 mg to 80 mg per 100 ml of the food product.

8. A method of using the composition for producing the anti-allergic agent according to claim 1 as an anti-allergic enhancer.

9. A method for producing a composition for producing an anti-allergic agent, comprising the steps of:
adding a solvent to tea leaf powders of one of green tea and pouchong tea made from Assam hybrid leaves to obtain a mixture; and
extracting a composition for producing an anti-allergic agent represented by the following general formula (1) from the mixture to purify the mixture by high-performance liquid chromatography: 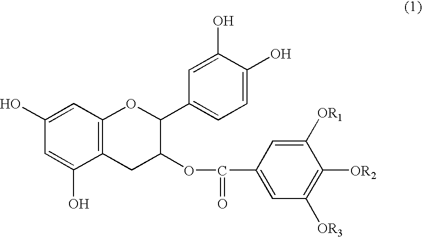
wherein R₁, R₂ and R₃ independently represent one of a hydrogen atom and a methyl group.

10. A method for using an octadecyl silica column having an inner diameter of 4.6 mm, a length of 150 mm, and a particle size of 5 μm to produce a composition for producing an anti-allergic agent represented by the following general formula (1): 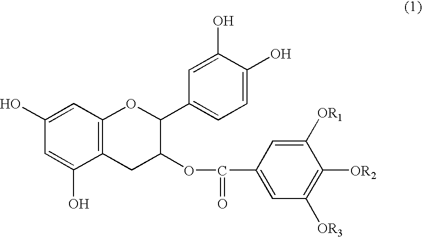
wherein R₁, R₂ and R₃ independently represent one of a hydrogen atom and a methyl group.

11. A beverage comprising the composition for producing the anti-allergic agent according to claim 2, in an amount in a range of 0.08 mg to 80 mg per 100 ml of the beverage.

12. A beverage comprising the composition for producing the anti-allergic agent according to claim 3, in an amount in a range of 0.08 mg to 80 mg per 100 ml of the beverage.

13. A beverage comprising the composition for producing the anti-allergic agent according to claim 4, in an amount in a range of 0.08 mg to 80 mg per 100 ml of the beverage.

14. A food product comprising the composition for producing the anti-allergic agent according to claim 2 in an amount of 0.08 mg to 80 mg per 100 ml of the food product.

15. A food product comprising the composition for producing the anti-allergic agent according to claim 3 in an amount of 0.08 mg to 80 mg per 100 ml of the food product.

16. A food product comprising the composition for producing the anti-allergic agent according to claim 4 in an amount of 0.08 mg to 80 mg per 100 ml of the food product.

17. A method of using the composition for producing the anti-allergic agent according to claim 2 as an anti-allergic enhancer.

18. A method of using the composition for producing the anti-allergic agent according to claim 3 as an anti-allergic enhancer.

19. A method of using the composition for producing the anti-allergic agent according to claim 4 as an anti-allergic enhancer.
